(19) United States
(12) Patent Application Publication
(10) Pub. No.: US 2016/0310065 A1
(43) Pub. Date: Oct. 27, 2016

(54) SPINAL CORD POSTURE MONITORING SYSTEM IN ANTERIOR/POSTERIOR AND LATERAL DIRECTIONS

(71) Applicant: UMM AL-QURA UNIVERSITY, Makkah (SA)

(72) Inventor: Muhammad ARIF, Makkah (SA)

(73) Assignee: UMM AL-QURA UNIVERSITY, Makkah (SA)

(21) Appl. No.: 14/696,086

(22) Filed: Apr. 24, 2015

(51) Int. Cl. A61B 5/00; A61B 5/11

(52) U.S. Cl. CPC A61B 5/4566; A61B 5/1118; A61B 5/1116; A61B 5/0022; A61B 5/7455; A61B 5/6823; A61B 2503/40; A61B 2503/10

(57) ABSTRACT

A spinal cord posture monitoring system for monitoring posture conformities of a spinal cord includes a wearable measurement device that houses a plurality of sensors, at least one mechanical assembly segment, the mechanical assembly segment includes a spherical encoder, a connector link connected to the spherical encoder on one end and to a hollow tube on the opposite end, such that the hollow tube is connected to the connector link on one end. The system also includes a communication circuit to communicate sensor information to a mobile application on a mobile device.

### BACKGROUND

The present disclosure relates generally to systems and methods for monitoring spinal cord posture in anterior/posterior and lateral directions and recommendations to remedy bad posture and related issues.

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Correct posture during performing different activities is very important for a healthy back. The spinal cord remains supported and stabilized when the back is kept straight and up right in the proper stance. Poor posture may cause many health problems, including back pain. Thus it is important to correct the bad posture of the spinal cord during normal day routines by guided rehabilitation or exercises.

Conventional back posture measuring and training devices have been known and used for many years. Most conventional back posture measuring and training devices suffer from various disadvantages in that they attempt to correct back posture by attempting to correct only the relationship (e.g., orientation) of the various segments of the spine relative to one another, while ignoring the orientation of the whole spine in relation to the rest of the body and more specifically in relation to the neck and in relation to true earth vertical. In other words, the segments of the spine may have correct relationships with respect to one another, but the entire spine may be at an incorrect angle leaning forward, backward, or be at one side while the individual segments maintain their respective relationships. Most conventional back posture training devices would not infer as bad posture if the segments of the spine maintain the correct relationships while the whole of the spine is leaning forward, backwards or is at one side. This could result in chronic sharp back pain.

Accordingly, in light of the current state of the art and the drawbacks to current conventional back posture training devices, a need exists for a posture training device that would correct posture in relation to multiple reference points as well as document, manage and share a person's posture history for advanced analysis by professionals.

### SUMMARY

In certain implementations, a spinal cord posture monitoring system may include a wearable measurement device configured to house a plurality of mechanical assembly segments, wherein each one of the plurality of mechanical assembly segments includes a spherical encoder configured to detect a position parameter and a motion parameter about three mutual orthogonal axes, a connector link operatively connected to the spherical encoder on one end and connected to a hollow tube on an opposite end, wherein the hollow tube is may receive the connector link on one end and to be affixed on an opposite end to another one of the plurality of mechanical assembly segments, and a communication circuit that communicates position and motion parameters as posture data for each spherical encoder of the wearable measurement device to a mobile application on a mobile device. At least one spherical encoder is configured to map a location of at least one vertebrae of a spinal cord. The spinal cord posture monitoring system also includes at least one stretching sensor that captures stretching data of the wearable measurement device in an anterior and in a posterior plane.

The spinal cord posture monitoring system also includes a neck sensor that operatively connects to the wearable measurement device and measures neck motion and neck orientation with regard to the spinal cord. The communication circuit records incidents of a non-conformity for each mechanical assembly segment, and records a time stamp and a geographic location stamp associated with the incidents of non-conformity. The mobile device is may operate in an animal monitoring mode and a human monitoring mode. An activity for which conformity is measured and recorded is selectable on the mobile application. The activity includes stretching, sitting, sleeping and performing an athletic activity. The athletic activity further includes tennis, football, swimming volleyball and weight lifting. After selecting an activity, the mobile application outputs a capability to download default settings from a remote location or record default manual settings input by a user. Default manual settings include at least one set of posture data, associated with the select activity, for the at least one of the mechanical assembly segments. The spinal cord posture monitoring system compares recorded default manual settings to downloaded default settings to measure accuracy of recorded default manual settings.

The mobile application is provides a review of the number of non-conforming violations recorded by the comfort level monitoring system within a predetermined period of time. The mobile application automatically transmits a notification to a medical facility indicating an occurrence of a non-conforming posture incident. The mobile application suggests corrective measures to correct the non-conforming posture incident. The corrective measures include a list of medication recommendations, a list of changes in posture, or initiating direct communications with the medical facility. The spinal cord posture monitoring system may also include a vibrating sensor that vibrates at a location where posture non-conformity is detected. The mobile application displays non-conforming activities to provide an alert of the non-conforming activities.

In other implementations, a spinal cord posture monitoring method may include placing a sensor for each vertebrae of a spinal cord to mimic an alignment of the spinal cord, selecting an activity to be performed by a person, determining default conformity settings for the selected activity, generating a conformity index for the selected activity based on a comparison between measured alignment data at each sensor and the default conformity settings, generating a time stamp and location stamp report indicative of measured non-conformity incidents, and generating an alert to remedy the non-conformity. The activity includes stretching, sitting, sleeping and performing an athletic activity.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a sample spinal cord posture monitoring system configuration according to an exemplary embodiment;

FIG. 2 is a sample mechanical sensor configuration within the spinal cord posture monitoring system according to an exemplary embodiment;

FIG. 3 is a system overview of a data logger, transmission and pre-processing system and a given interaction configuration with different sensor blocks according to an exemplary embodiment;

FIG. 4A is a sample mechanical sensor configuration for integrated extended neck posture monitoring systems according to an exemplary embodiment;

FIG. 4B is an illustration of good and poor neck posture measurement in relation to the spinal cord according to an exemplary embodiment;

FIG. 5 is an illustration of different bending angles and measurement of different bending angles according to an exemplary embodiment;

FIG. 6 is a sample graphical user interface displaying an initial listing of profiles for application of the spinal cord posture monitoring system according to an exemplary embodiment;

FIG. 7A is a sample graphical user interface displaying stretching activity options for a human associated with the spinal cord posture monitoring system according to an exemplary embodiment;

FIG. 7B is a sample stretching activity options for a human associated with the spinal cord posture monitoring system according to an exemplary embodiment;

FIG. 8A is a sample graphical user interface displaying sitting activity options for a human associated with the spinal cord posture monitoring system according to an exemplary embodiment;

FIG. 8B is a sample sitting activity options for a human associated with the spinal cord posture monitoring system according to an exemplary embodiment;

FIG. 9A is an illustration of a functional menu for measuring status and compliance of an individual wearing the spinal cord posture monitoring system while performing an athletic activity, according to an exemplary embodiment;

FIG. 9B is an illustration of a functional menu for downloading default settings to measure status and compliance of an individual wearing the spinal cord posture monitoring system according to an exemplary embodiment;

FIG. 9C is an illustration of a functional menu for inputting default settings to measure status and compliance of an individual wearing the spinal cord posture monitoring system according to an exemplary embodiment;

FIG. 9D is an illustration of a functional menu for tracking conformity index to measure status and compliance of an individual wearing the spinal cord posture monitoring system according to an exemplary embodiment;

FIG. 9E is an illustration of a functional menu for reviewing the number of violations to measure status and compliance of an individual wearing the spinal cord posture monitoring system according to an exemplary embodiment;

FIG. 9F is an illustration of a functional menu for reviewing the number of transmitted doctor pings to measure status and compliance of an individual wearing the spinal cord posture monitoring system according to an exemplary embodiment;

FIG. 9G is an illustration of a functional menu for outputting suggested corrective measures for an individual wearing the spinal cord posture monitoring system according to an exemplary embodiment;

FIG. 9H is an illustration of a functional menu for suggesting exercises for an individual wearing the spinal cord posture monitoring system according to an exemplary embodiment;

FIG. 10 is an algorithmic flow diagram for measuring status and compliance method 1000 of an individual wearing the spinal cord posture monitoring system according to an exemplary embodiment; and

FIG. 11 is an illustration of a hardware diagram of a device according to exemplary embodiments.

### DETAILED DESCRIPTION

The detailed description set forth below in connection with the appended drawings is intended as a description of presently preferred embodiments of the disclosure and is not intended to represent the only forms in which the present disclosure may be constructed and or utilized.

For purposes of illustration, programs and other executable program components are illustrated herein as discrete blocks, although it is recognized that such programs and components may reside at various times in different storage components and are executed by the data processor(s) of the computers. Further, each block within a flow chart may represent method function(s), operation(s), or act(s) and one or more elements for performing the method function(s), operation(s), or act(s). In addition, depending upon the implementation, the corresponding one or more elements may be configured in hardware, software, firmware, or combinations thereof.

For the sake of convenience and clarity, the disclosure defines the terms posture as the body position as a whole at a given moment. Further, and also for the sake of convenience and clarity, the disclosure defines good posture as the proper or appropriate body position as a whole at a given moment for a given activity, such as proper posture for the exemplary, non-limiting activities of standing, sitting, walking, jumping kneeling, or proper posture for various athletic activities, such as basketball free-throw shots, tennis service motions, golf swings, aerobics, or yoga. It should be noted that references to human body, and in particular, human vertebra throughout the disclosures is meant as illustrative and for convenience of example, only. The present disclosure may be used for training proper posture for non-human applications as well, such as dogs or other animals, and may also be used for proper posture training for proper use of hands, arms, or legs for a particular activity and therefore, is not limited to humans or human vertebrae.

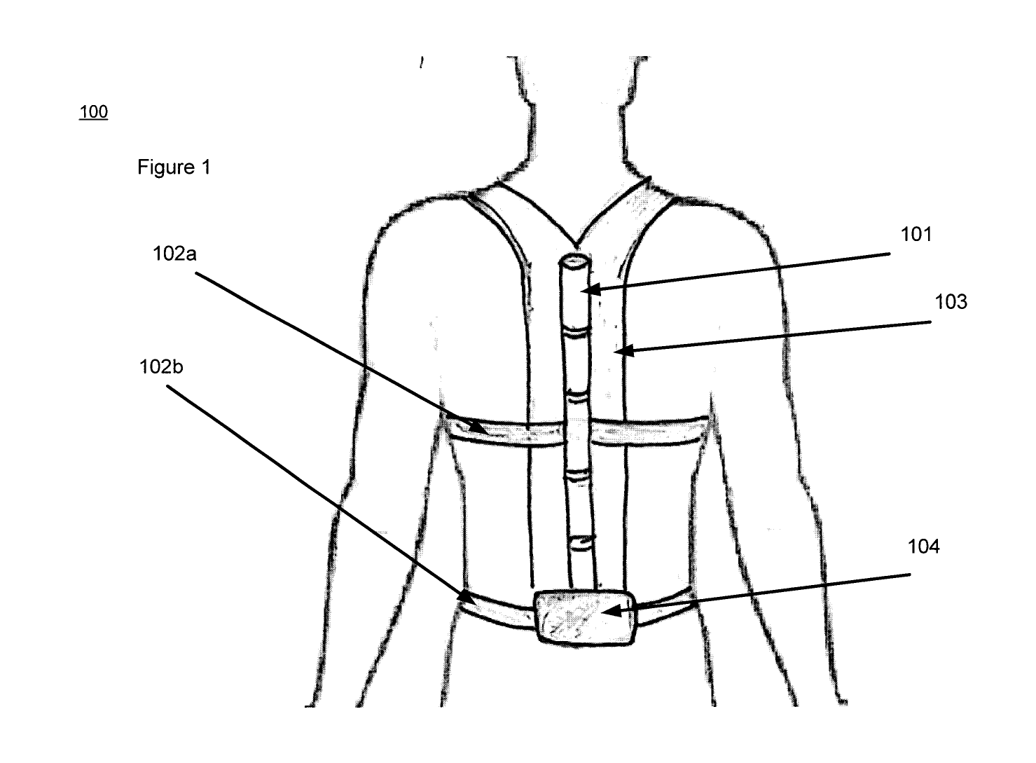

FIG. 1 illustrates a sample spinal cord posture monitoring system (SCPMS) 100 configuration according to an exemplary embodiment. SCPMS 100 includes a bending pattern measurement device 101, which can be attached to the body via strap assemblies 102a, 102b and 103 and data collection and transmission module 104. Strap assemblies 102a, 102b and 103 can be made into multiple configurations that best suit an individual using SCPMS. For example, in one configuration, strap assemblies 102a and 102b can be a single strap (such as 102a) that surrounds a mid-section segment of a user or can be made to be double straps; one strap to secure the assembly on the user's mid-section (102a) while the other strap is used to secure the assembly on the user's waist line (102b). Strap assembly 103 can be made of a longitudinal configuration designed to secure SCPMS along the user's back. Strap assembly 103 is designed to further secure SCPMS by connecting to strap assemblies 102a and 102b on the back side and front side of the user. For example, Strap assembly 103 connects to strap assembly 102a at the mid-section and strap assembly 102b at the waist line section on the back side. On the front side, strap assembly 103 may connect to strap assembly 102a at the mid-section by going over the shoulders of the user. Of course, different combinations of strap attachments, alignments, and placements may be arranged in any one of different formations including integration within items of clothing such as T-shirts, sports shirts, jumpers, sweaters and other clothing items as one of ordinary skill in the art would recognize.

Data collection and transmission module 104 collects, records, and transmits all data related to bending motions of the user. Data collection and transmission module 104 is described in greater detail below.

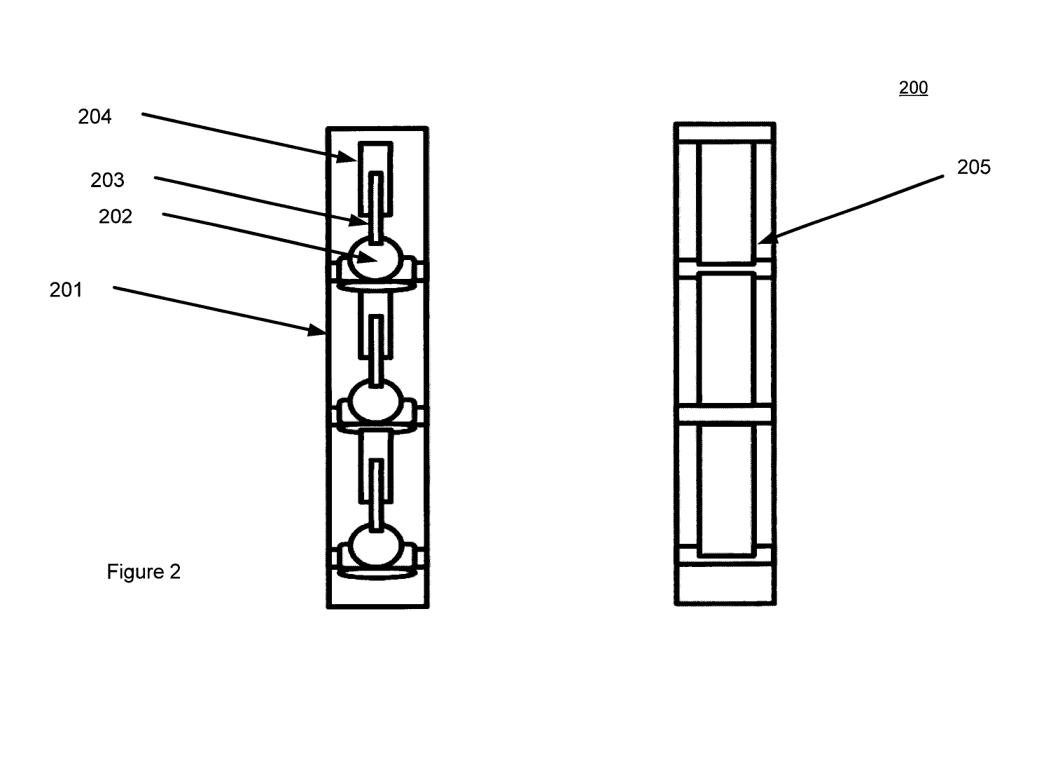

FIG. 2 is a sample mechanical sensor configuration 200 within the SCPMS according to an exemplary embodiment. Mechanical sensor configuration 200 includes a series of mechanical assemblies fixed in a tube 201. Each mechanical assembly includes spherical encoder 202, connector stick 203, hollow tube 204 and stretching sensors 205. Spherical encoder 202 is designed to provide the motion of stick 203 in lateral as well as anterior/posterior directions. Spherical encoder 202 includes a spherical rotor, stator (not shown) and sensor assemblies (not shown) that measure the rotation angles of the spherical rotor. Connector stick 203 connects to and engages hollow tube 204 that is affixed to the mechanical assembly above. For example, mechanical assembly 201A includes connector stick 203 that connects to and engages hollow tube 204 that is affixed to mechanical assembly 201B. The length of hollow tube 204 is designed to be sufficient to allow the stick to freely move within the tube but also to remain in the hollow tube in cases where a full bending motion occurs.

Data from the spherical encoder 202 for each assembly is transmitted to data collection and transmission module 104. The data can be transmitted via wire connection or a wireless connection, including but not limited to, WiFi and Bluetooth. Each mechanical assembly further includes stretching sensors 205 attached between the fixed points of the mechanical assembly. Stretching motions of stretching sensors 205 in the anterior and posterior planes are recorded from each stretching sensor in both directions. The motions are transmitted to data collection and transmission module 104 for collection, documentation and further analysis.

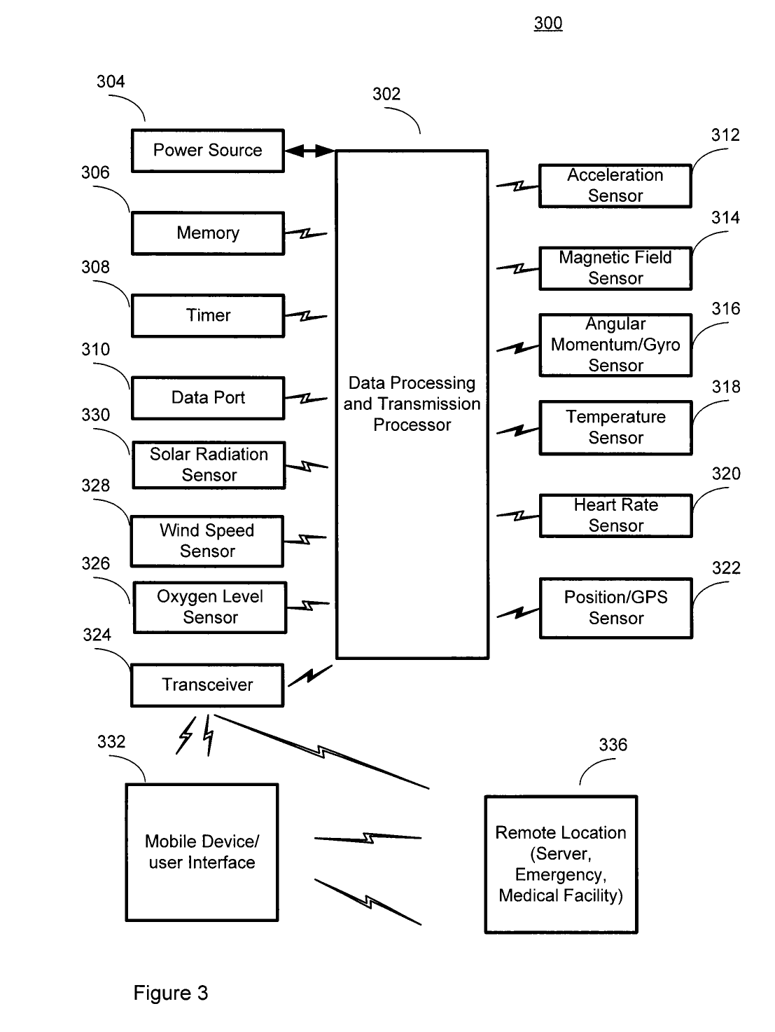

FIG. 3 is an exemplary overview of data collection and transmission module assembly 300 and a given interaction configuration with different sensor blocks according to an exemplary embodiment. Data collection and transmission module assembly 300 includes data processing and transmission processor 302, a power source 304, memory 306, timer 308, data port 310, acceleration sensor 312, magnetic field sensor 314, angular momentum sensor 316, temperature sensor 318, heart rate sensor 320, position/GPS sensor 322 and transceiver 324, oxygen level sensor 326, wind speed sensor 328, and solar radiation sensor 330.

The power source 304 provides power to the data processing and transmission processor 302. In one embodiment, the power source 304 may be a battery. The power source may also be built into the data processing and transmission processor 302 or removable from the data processing and transmission processor 302, and may be rechargeable or non-rechargeable. In an exemplary embodiment, the power source 304 may be recharged by a cable attached to a charging source, such as a universal serial bus (“USB”), FireWire, Ethernet, Thunderbolt, headphone cable, or a specially designed cable attached to a personal computer. The power source 304 may also be recharged by inductive charging, which uses an electromagnetic field to transfer energy from an inductive charger to the power source 304 when the two are brought in close proximity. Thus, the power source need not be plugged into one another via a cable. A docking station may also be used to facilitate charging.

The memory 306 may store application program instructions and store default and updated posture and motion activity data. In an embodiment, the memory 306 may store application programs used to implement aspects of the functionality of the spinal cord posture monitoring system described herein. The memory 306 may be a Random Access Memory (RAM), Read Only Memory (ROM), programmable ROM (PROM), flash memory, or other types of data storage devices. The memory 306 may also store raw data, recorded data, and/or calculated data, downloaded data and/or as explained in further detail below, the memory 306 may act as a data storage buffer. The memory 306 may include both read only memory and random access memory, and may further include memory cards or other removable storage devices. The memory 306 may store data in memory locations of predetermined size, i.e., bytes, words, sectors, and/or blocks, such that only a certain quantity of data may be saved for a particular application of the current data processing and transmission processor 302.

The timer 308 may be a clock that tracks absolute time and/or determines elapsed time. In some exemplary embodiments, the timer 304 may be used to timestamp certain data records, such that the time that certain data was measured or recorded may be determined and various timestamps of various pieces of data may be correlated with one another.

The data port 310 facilitates information transfer to and from the data processing and transmission processor 302 and may be, for example, a USB port. The data port 310 can additionally or alternatively facilitate power transfer to power source 304, in order to charge power source 304.

The acceleration sensor 312 measures the acceleration of the data processing and transmission processor 302 when it is placed on the individual or the acceleration of sensors on clothing items worn by the individual. For example, if data processing and transmission processor 302 is physically coupled to an object, such as a clothing item or any item placed on an individual (such as a T-shirt, not shown), the acceleration sensor 312 may measure the acceleration of the individual, including the acceleration due to the earth's gravitational field. In one exemplary embodiment, the acceleration sensor 312 may include a tri-axial accelerometer that measures acceleration in three orthogonal directions. Of course two, three, or more separate accelerometers may be used in the alternative without departing from the scope of the present disclosure.

The magnetic field sensor 314 measures the strength and direction of magnetic fields in the vicinity of the data processing and transmission processor 302. Accordingly, when the data processing and transmission processor 302 is physically coupled to an individual or clothing item, the magnetic field sensor 314 may measure the strength and direction of magnetic fields in the vicinity of the individual, including the earth's magnetic field. In one exemplary embodiment, the magnetic field sensor 314 may be a vector magnetometer. The magnetic field sensor 314 may also be a tri-axial magnetometer that measures the magnitude and direction of a resultant magnetic vector for the total local magnetic field in three dimensions. Two, three, or more separate magnetometers may be used as one of ordinary skill would recognize.

In one example, the acceleration sensor 312 and the magnetic field sensor 314 may be contained within a single accelerometer-magnetometer circuit integrated circuity such as LSM303DLHC made by STMicroelectronics of Geneva, Switzerland. The data logger may include only one of the acceleration sensor 312 and the magnetic field sensor 314, and may omit the other if desired.

The angular momentum sensor 316, which may be a gyroscope, is adapted to measure the angular momentum or orientation of the data processing and transmission processor 302. Accordingly, when the data processing and transmission processor 302 is physically coupled to an individual or clothing item 100, the angular momentum sensor 316 may measure the angular momentum or orientation of the individual. The angular momentum sensor 316 may be a tri-axial gyroscope that measures angular rotation about three orthogonal axes. Two, three, or more separate gyroscopes may be used instead, however. In an exemplary embodiment, the angular momentum sensor 316 may be used to calibrate measurements made by one or more of the acceleration sensor 312 and the magnetic field sensor 314.

The temperature sensor 318 may be, for example, a thermometer, a thermistor, or a thermocouple that measures changes in the temperature. The temperature sensor 318 may be used for calibration of other sensors of the spinal cord posture monitoring system, such as the acceleration sensor 312 and the magnetic field sensor 316. The temperature sensor 318 may also be used to calculate temperature and humidity levels of the surrounding environment. Such temperature and humidity levels that can be further utilized to determine potential correlation between posture and environmental conditions as will be described further below.

The heart rate sensor 320 may measure the individual's heart rate and may be placed in contact with the individual's skin, such as on the chest or wrist, and secured with a strapping mechanism. Heart rate sensor 320 may provide an alarm signal if a person's heart rate falls below a given threshold or rises above a given threshold. For example, if a person's heart rate rises above 220 beats per minute (bpm) then the alarm signal is sent to the data processing and transmission processor 302. Alternatively, if a person's heart rate falls below 40 bpm, then the alarm signal is also sent to data processing and transmission processor 302. In another example, the predetermined threshold may be tailored for different personal attributes of the person wearing the sensor. For example, a different threshold may be designated for a man than for a woman. Alternatively, a different threshold may also be designated depending on age of the person and weight of the person.

The position/GPS sensor 322 may be an electronic satellite position receiver determines its location (i.e., longitude, latitude, and altitude) using time signals transmitted along a line-of-sight by radio from satellite position system satellites. Known satellite position systems include the GPS system, the Galileo system, the BeiDou system, and the GLONASS system. The position/GPS sensor 322 may also be an antenna that communicates with local or remote base stations or radio transmission transceivers to determine the location of data transmission circuit 302 using radio signal triangulation or other similar principles. The position/GPS sensor 332 data may allow data processing and transmission processor 302 to detect information that may be used to measure and/or calculate position waypoints, time, location, distance traveled, speed, pace, or altitude as would be recognized by one of ordinary skill.

The transceiver 324 enables data processing and transmission processor 302 to wirelessly communicate with other components of the spinal cord posture monitoring system 300, such as those described in further detail below. For example, the data processing and transmission processor 302 and the other local components of the spinal cord posture monitoring system 300 may communicate over a personal area network or local area network using, for example, one or more of the following protocols: ANT, ANT+ by Dynastream Innovations, Bluetooth, Bluetooth Low Energy Technology, BlueRobin, or suitable wireless personal or local area network protocols. Other known communication protocols suitable for a spinal cord posture monitoring system may also be used.

In one exemplary embodiment, transceiver 324 is a low-power transceiver and may be a two-way communication transceiver 324, or a one-way transmitter or a one-way receiver. Wireless communication between data processing and transmission processor 302 and other components of the spinal cord posture monitoring system 300 is described in further detail below. Alternatively, data processing and transmission processor 302 may be in wired communication with other components of the spinal cord posture monitoring system 300 and not rely on transceiver 324 as can be appreciated.

Other sensors may also be included in the system and may communicate with data processing and transmission processor 302. For example, the system may include oxygen level sensor 326, wind speed sensor 328 and solar radiation sensor 330 as part of a sensor suit that measures environmental factors that may attribute to posture misalignment.

Oxygen level sensor 326 measures oxygen levels in the surrounding areas where the individual is conducting a physical activity such as walking, running, hiking, or sitting. Oxygen level sensor 326 may be further used to measure oxygen level ratios within the surrounding environment, and may compare a ratio of existing oxygen levels to expected levels or normal oxygen levels for that environment. Oxygen level sensor may be any type of oxygen level sensors, including, for example, Honeywell oxygen sensor having 3 series of flange mounts and probe housing that contains two Zirconium Dioxide (ZrO2) discs with a small, hermetically sealed chamber between each disc. The ZrO2 technology provides oxygen measurement without reference gas, which results in enhanced accuracy and durability.

Wind speed sensor 328 may be an anemometer used to measure wind speed at a given location. Diverse types of anemometers may be deployed depending on their location of deployment. For example, cup and vane anemometers may be difficult to deploy within clothing items, but may be deployed throughout the geographic vicinity where the individual and crowd are expected to be gathered. A single anemometer may be used, or alternatively, a series of anemometers are used, each providing potentially different wind speeds for different locations to the spinal cord posture monitoring system 300. A hot wire anemometer may further be deployed within spinal cord posture monitoring assembly. A hot wire anemometer uses a very fine wire electrically heated up to some temperature above the ambient. Air flowing past the wire has a cooling effect on the wire. As the electrical resistance of most metals is dependent upon the temperature of the metal, a relationship can be obtained between the resistance of the wire and the flow speed. Of course, other anemometer types may also be used without departing from the scope of the present disclosure. Thus, the anemometers described herein are merely exemplary and not limiting upon the disclosure.

Solar radiation sensor 330 determines the available solar energy radiation and may include a light sensing element that detects the quantity or intensity of solar radiation and converts the quantity or intensity into an electric signal. The electric signal is sent to data processing and transmission processor 302 to determine solar radiation exposure and intensity, which is a parameter that may be used in determining the correlation between posture patterns of an individual. An example of a solar radiation sensor can be a pyranometer that is used to measure the solar radiation flux at a given time from a field of view of 180 degrees. The pyranometer does not require any power to operate, thus reducing the potential energy consumption of the spinal cord posture monitoring system 300.

Once data processing and transmission processor 302 collects all sensor data associated with an individual or a group of individuals, such as a single family, or a group of friends all selecting to monitor their posture, or a selected group for a study, or a grouping of dispirit individuals admitted to a health clinic for treatment, etc., the data may be forwarded to mobile device/user interface 332. Mobile device/user interface 332 may process and house a variety of information for dissemination to multiple user interface devices in remote locations, such as a server, emergency medical facility, or another mobile device/user interface 336. For example, mobile device/user interface 332 can process the data received and display posture related information to the user. Mobile device/user interface 332 can also share the information with other mobile device if prompted by a user, or share information with a remote server or medical or emergency facilities either if prompted by user or automatically in cases of measured emergencies.

Spinal cord posture monitoring system 300 may further include features that enable the mobile device/user interface and/or data processing and transmission processor 302 to interact with web based systems to retrieve individual related statistics. For example, data processing and transmission processor 302 may connect to and process data from a single data transmission circuit or a specified number of data transmission circuits associated with a given number of individuals. These individuals can be placed in groups or can be randomly deployed to determine spinal cord posture settings in different environments and at different locations within given geographic vicinity. However, in order to perform specific data manipulations and comparisons on a geographic vicinity wide basis, data processing and transmission processor 302 and or mobile device 332 may connect to internet based systems to download other individual/group information. This is useful in determining activity based default posture settings for each downloadable activity. In one example, data processing and transmission processor 302 may communicate with multiple data transmission circuits deployed on individuals.

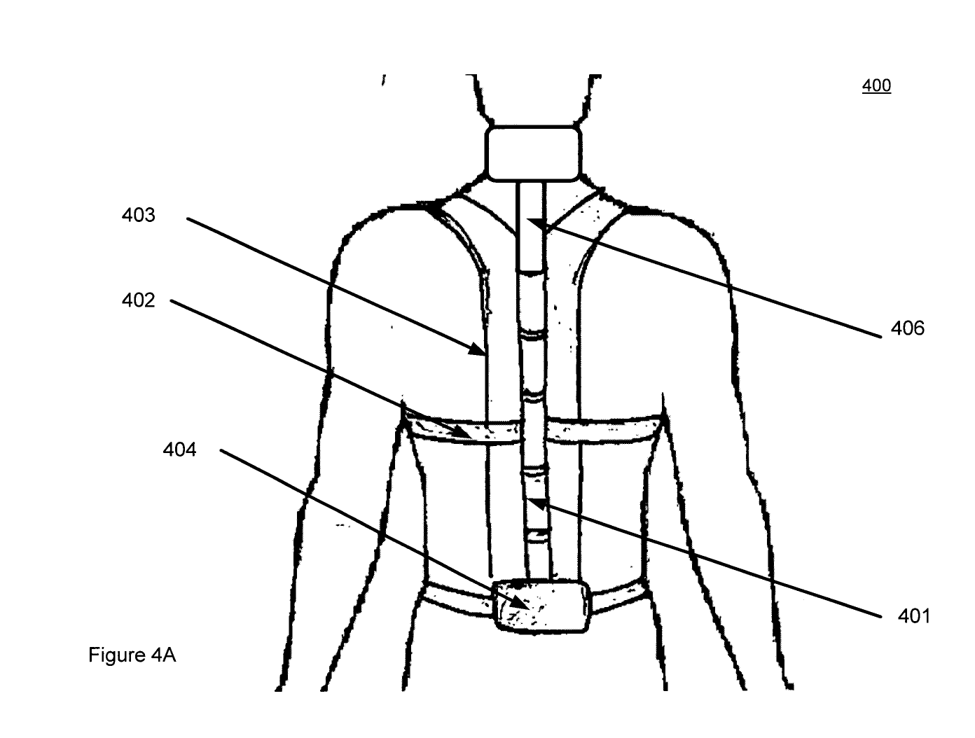

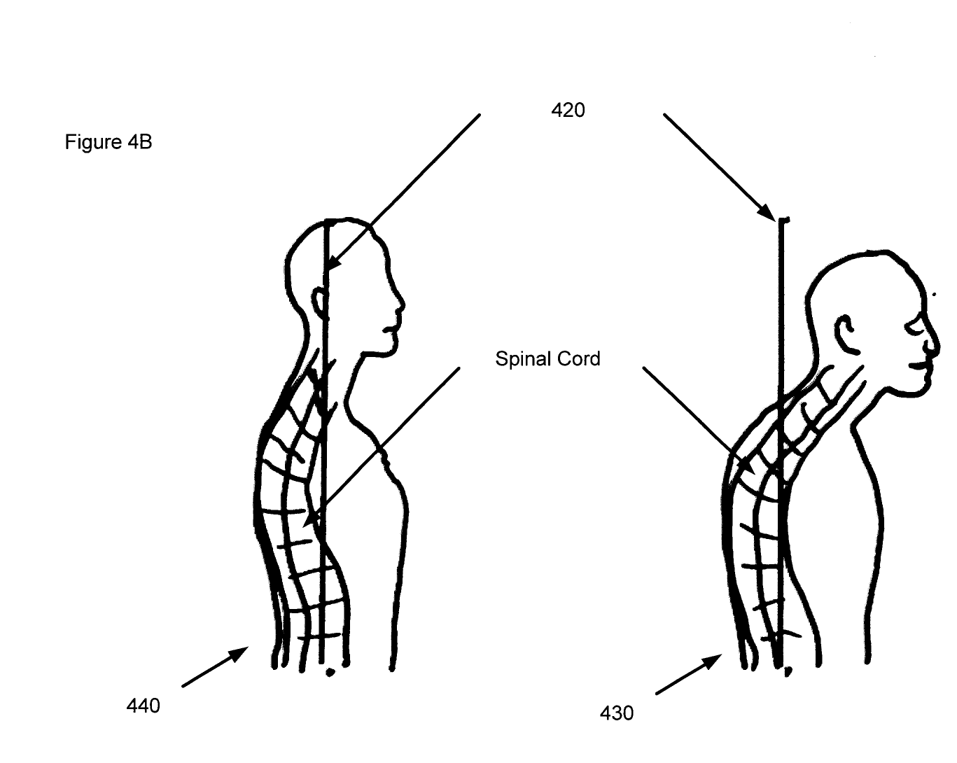

FIG. 4A is a sample mechanical sensor configuration for SCPMS 400 with integrated extended neck posture monitoring systems according to an exemplary embodiment. SCPMS 400 includes bending pattern measuring device 401, which is attached to the body via strap assemblies 402 and 403 or through alternative means previously discussed, such as T-shirts, or other clothing attires, data collection and transmission module 404, and neck collar module 406 for measuring of bending patterns of an individual's neck with respect to the back. The additional neck information is useful for a medical specialist diagnosing reasons for neck pain and other spinal cord pains related to neck movement and neck postures. A medical specialist can monitor neck movements and postures during rehabilitation periods for example. Neck collar module 406 may be integrated within SCPMS 400 by attaching to pattern measuring device 401. Pattern measuring device 401 includes a plurality of mechanical assembly segments that include connector link, spherical encoder and a hollow tube to measure the bending motions. In one example, Neck collar module 406 includes a connector tube that is connected to a spherical encoder on one end and a hollow tube on the other. The connector tube is inserted within the hollow tube to allow for sufficient bending measurement without detaching from the hollow tube by an exit motion. The neck collar module 406 spherical encoder measures the movement of the neck with respect to the pattern measuring device 401. Because the pattern measuring device 401 measures the movement of the spinal cord, the neck collar module 406, therefore, measures the movement of the neck with respect to the spinal cord.

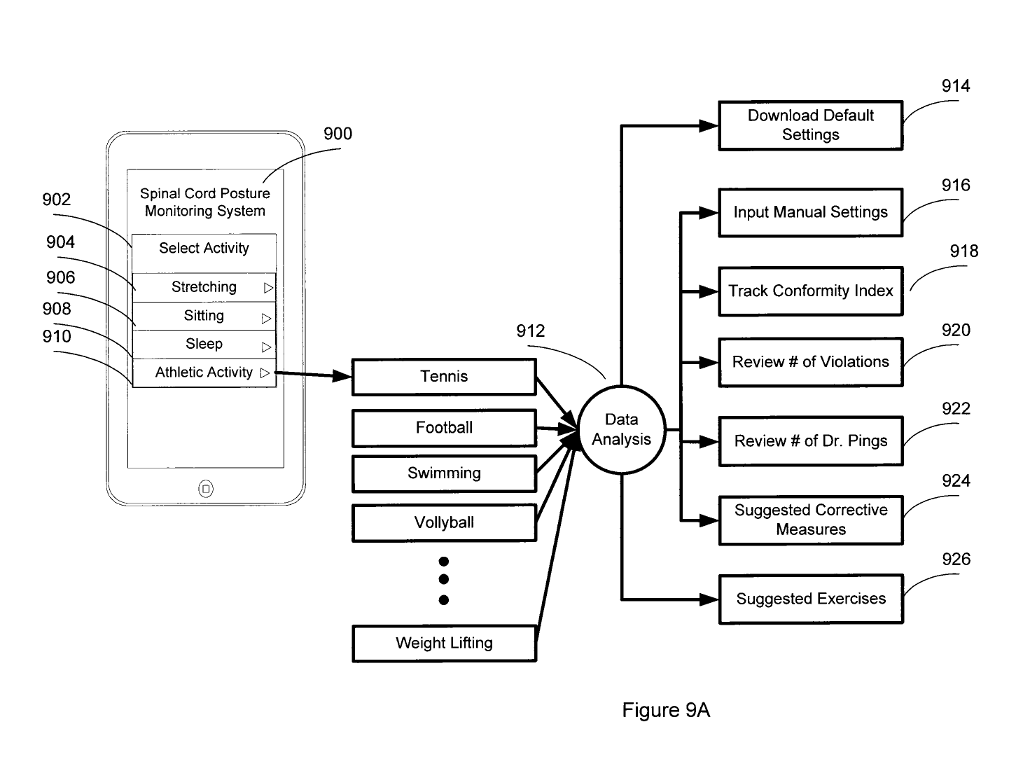

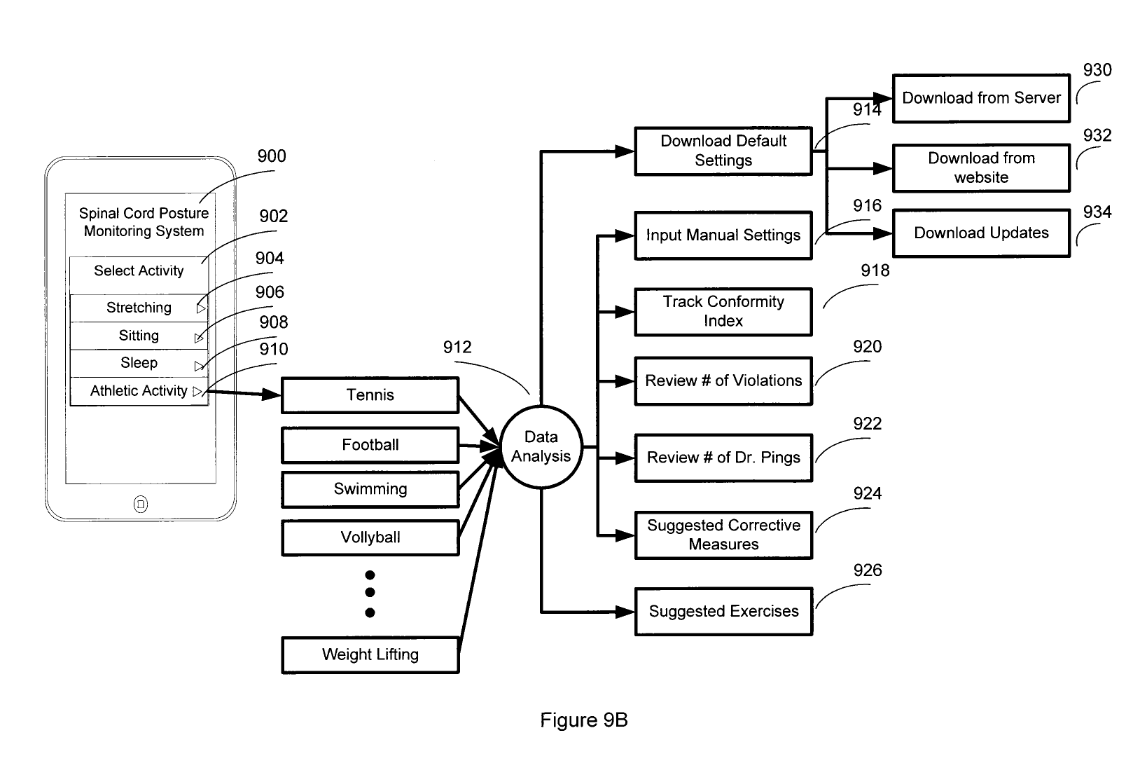

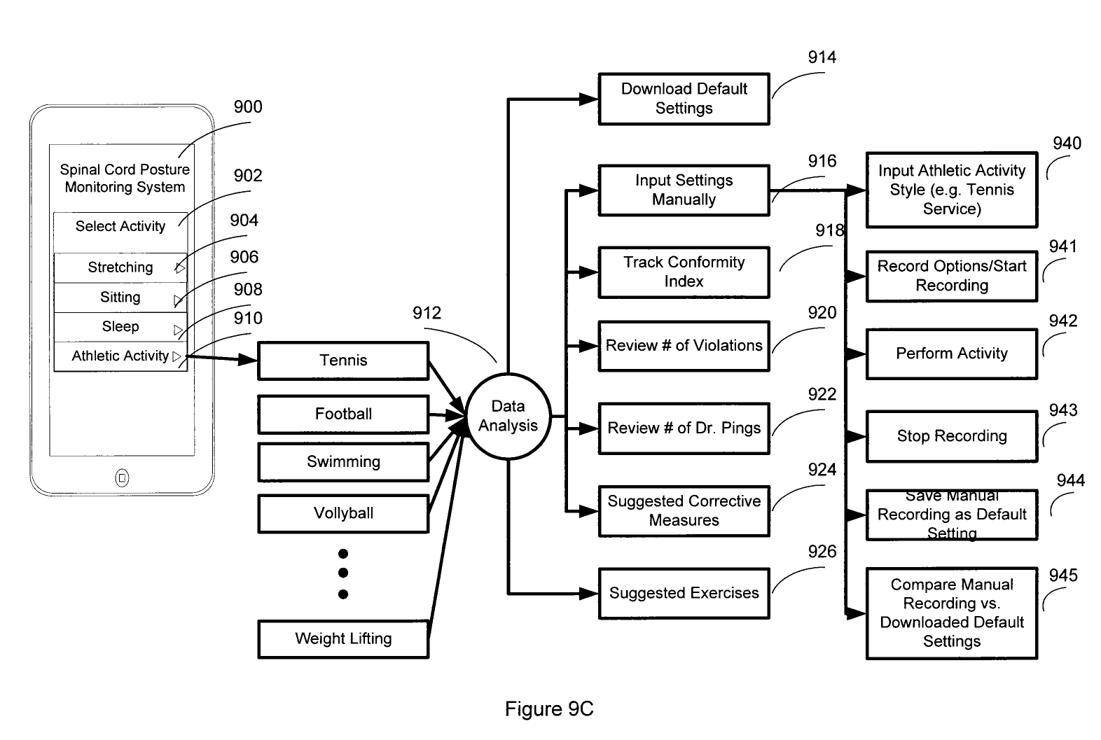

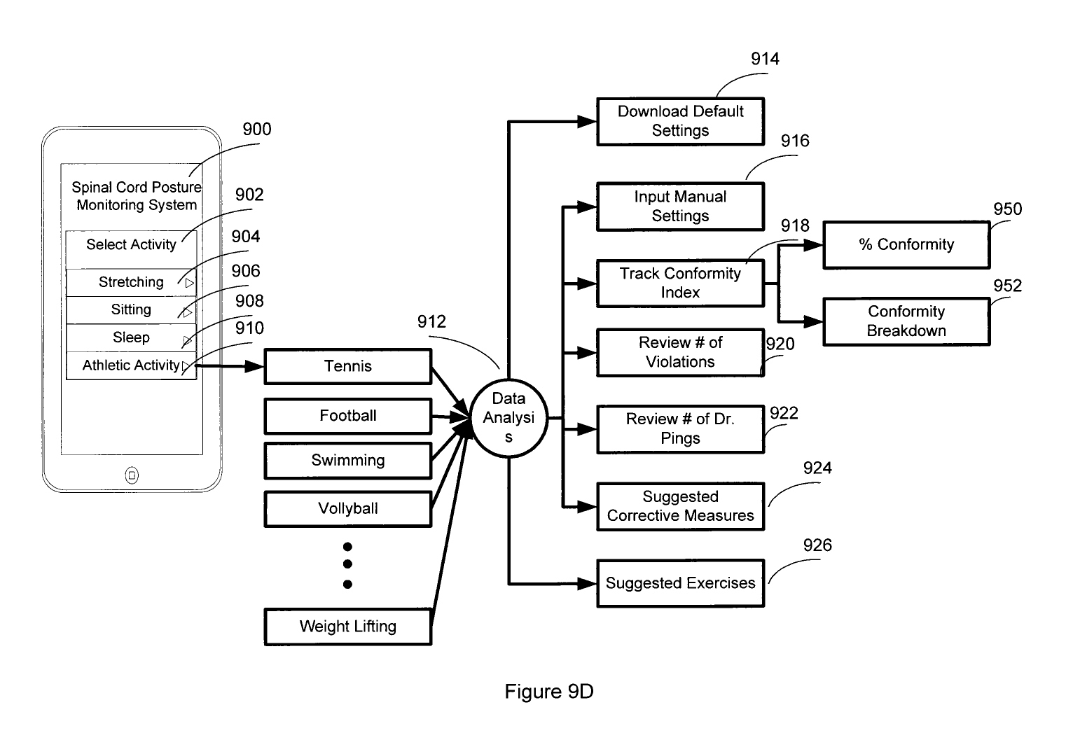

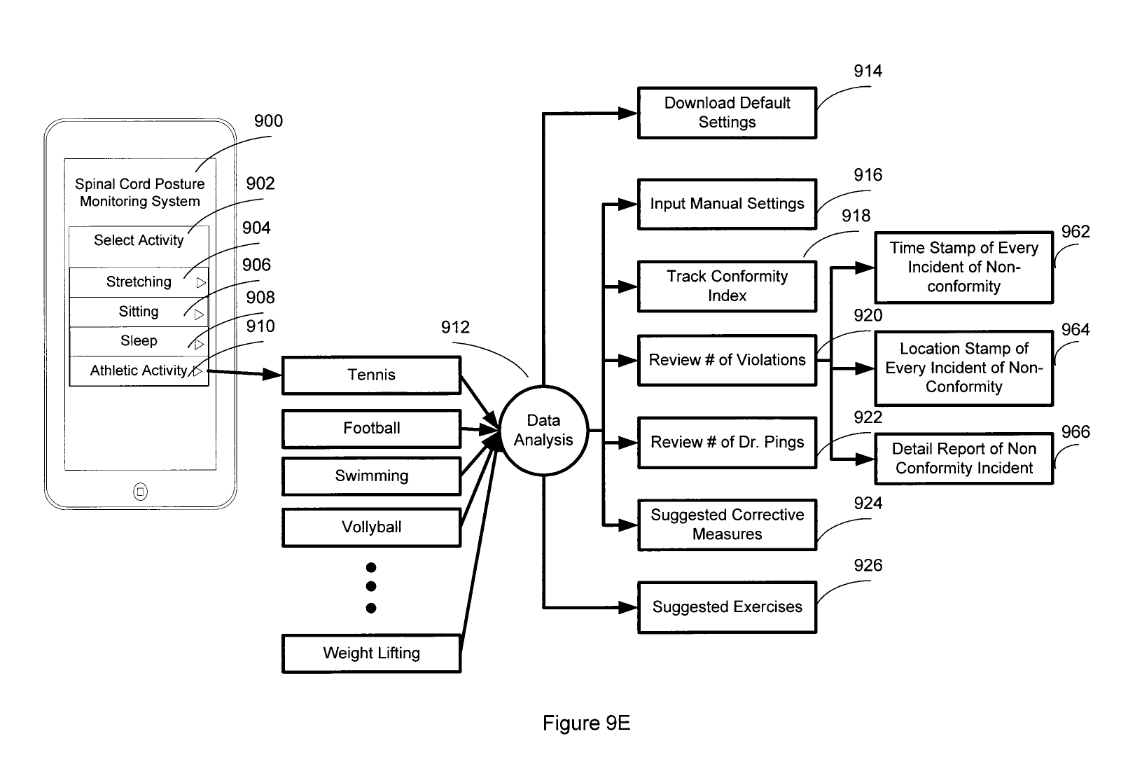

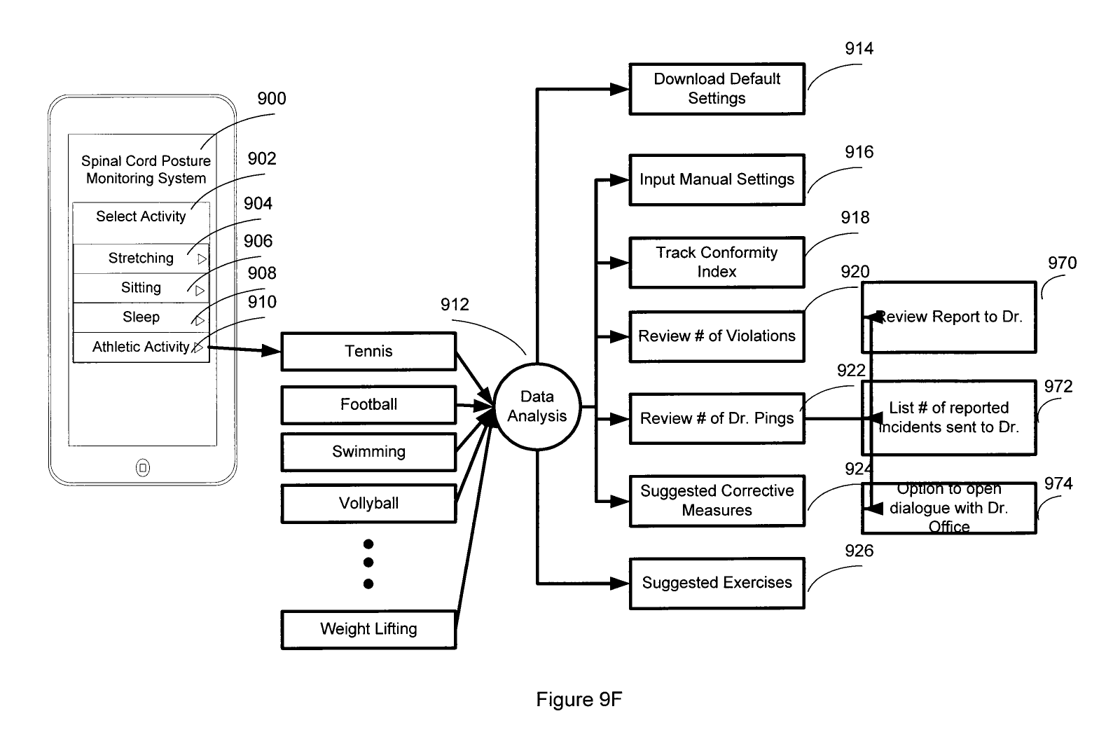

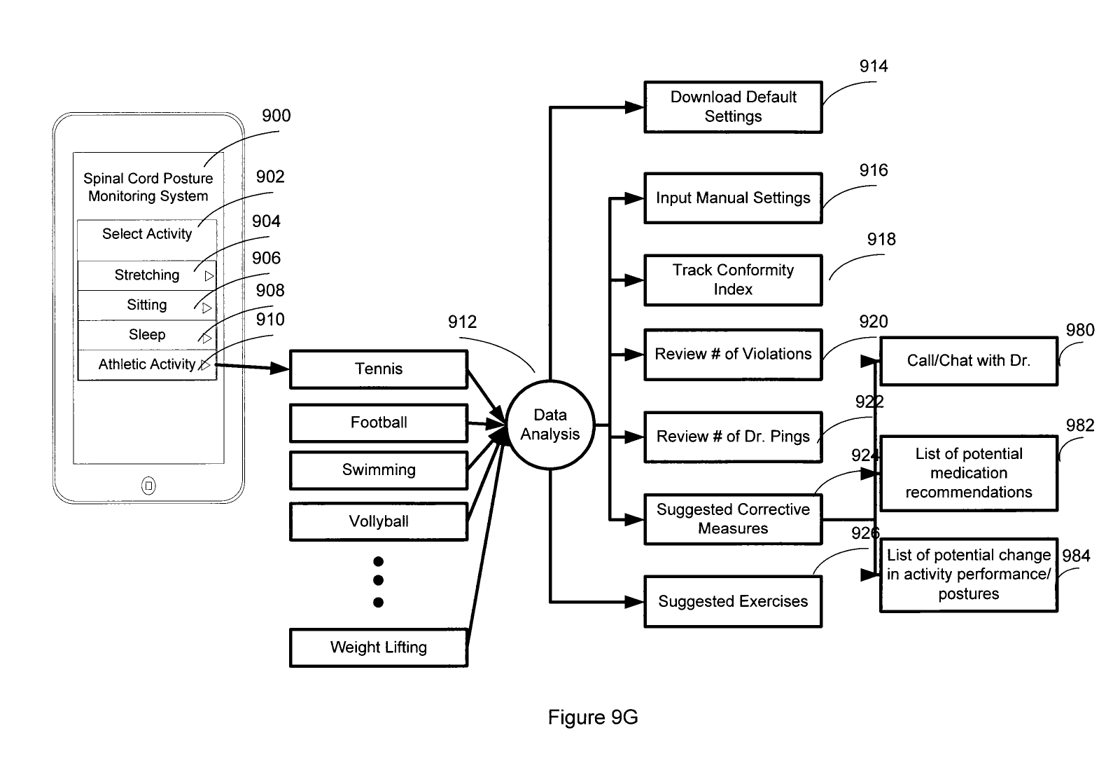

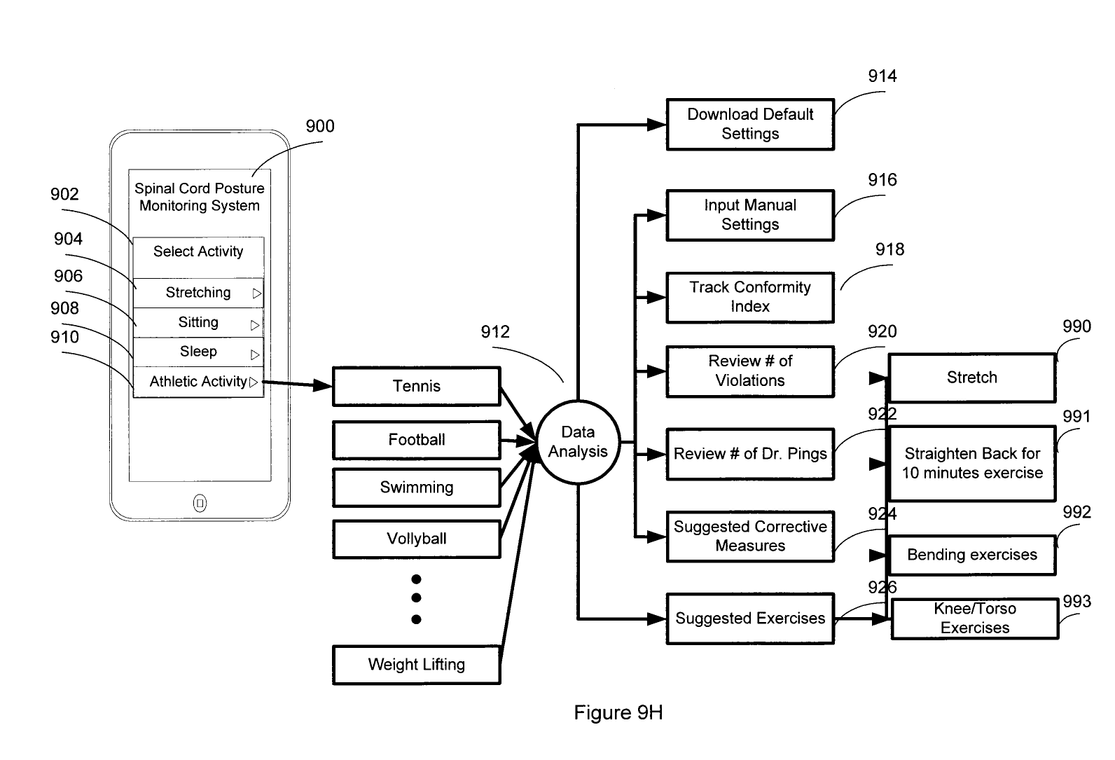

FIG. 4B is an illustration of good and poor neck posture examples and measurement in relation to the spinal cord according to an exemplary embodiment. Poor neck posture 430 may result in forward head posture (FHP) which can result due to long use of computer equipment or due to prolonged driving habits or poor sleeping postures. Poor neck posture 430 may cause neck, head and shoulder pain. FHP causes pressure on the nerves which causes headaches and as well as excessive weight stress on the cervical spine. Poor neck posture 430. Line 420 extends from the center of the external auditory meatus (EAM) and dropped down in the correct posture is illustrated. When a head is perfectly balanced on this line, as in good posture illustration 440. An FHP as illustrated by poor neck posture 430 shows an extreme curvature of the spinal cord. This may be further mapped by SCPMS 400 on a mobile device for a user to view a mapping of the user's posture and ways to correct. Examples of the mobile device and SCPMS application are further illustrated in FIGS. 9A-9H.

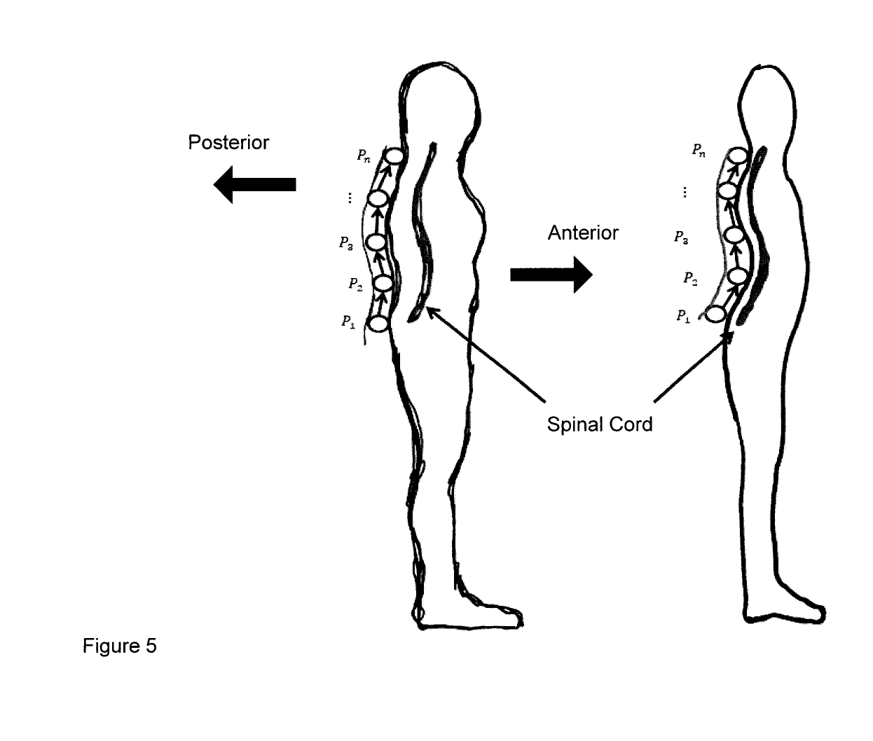

FIG. 5 is an illustration of different bending angles and measurement of different bending angles according to an exemplary embodiment. Bending angles are measured from the placed mechanical assemblies illustrated as data points P₁, P₂, . . . , Pₙ displaced alongside the back to mimic the spinal cord to give angles in anterior/posterior and lateral directions. An interpolation method is used to get the continuous curvature of the back and associated spinal cord in a particular posture in both anterior/posterior directions. In one example, cubic spline interpolation is used interpolate the curvature of the back. Cubic spline interpolation gives a function which is continuous in both the first and second order derivatives and passes through all the data points of interest. For example, the data points of interest are P₁, P₂, P₃, . . . , P_{N} of FIG. 5 which are measured and tabulated by SCPMS 400.

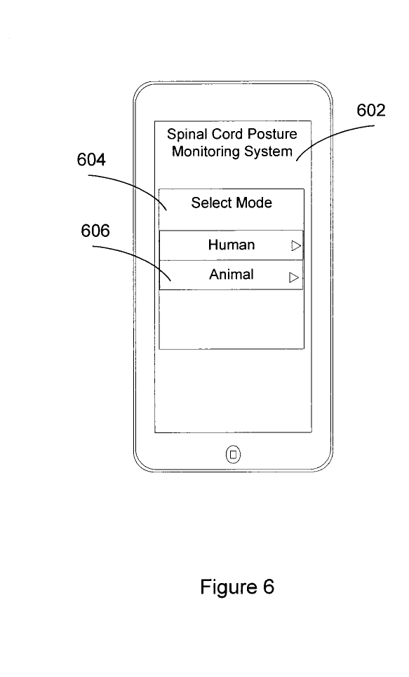

FIG. 6 is a sample graphical user interface displaying an initial listing of profiles for application of the spinal cord posture monitoring system according to an exemplary embodiment. SCPMS graphical user interface include SCPMS configuration module 602. Configuration module 602 allows a user to initially select a detection/analysis mode 604. For example, a user can apply SCPMS to themselves or to a pet. If a user wants to apply SCPMS to themselves, the user selects human option 606 to enter human configuration control. If a user wants to apply SCPMS to their pet, the user selects animal option 608 to enter animal configuration control. Animals may also need spinal cord posture measurement and correction measures. Selecting animal option 608 allows a user to manage a large selection of option relating to configuring, recording, and reporting spinal cord posture measurements and to download potential corrective measures. For the sake of illustration, examples of section of human option 606 will be described in greater detail. However, the options can be similarly applied to animal modes as one of ordinary skill in the art will recognize.

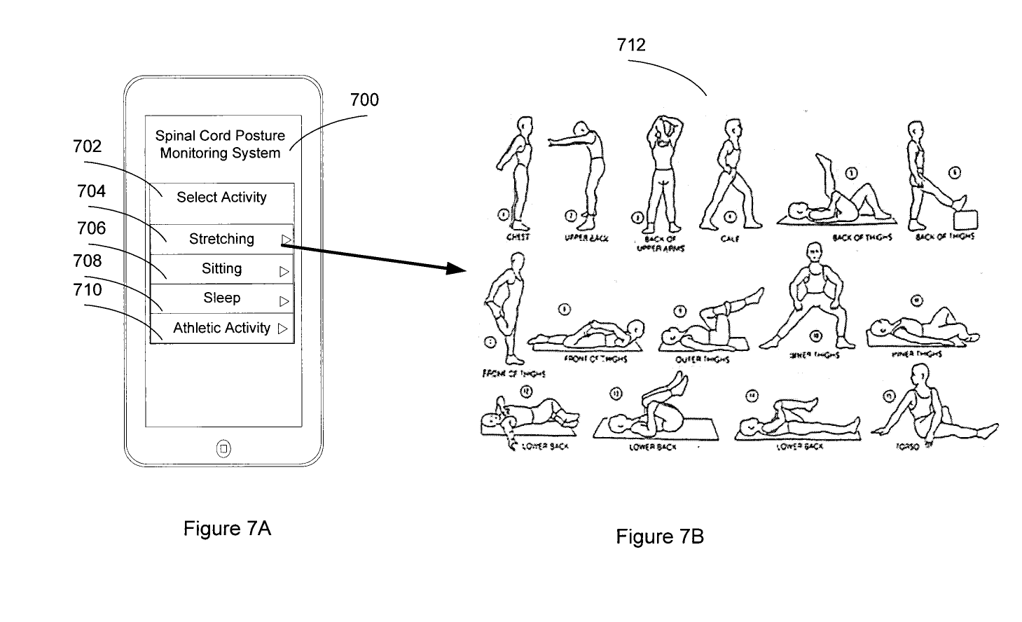

FIG. 7A is a sample graphical user interface displaying stretching activity options for a human associated with the spinal cord posture monitoring system according to an exemplary embodiment. FIG. 7B is a sample display of stretching activity options for a human associated with the spinal cord posture monitoring system. Most people, either through training or naturally, perform an activity with the appropriate posture for that activity when they consciously realize that their body has an incorrect posture for the activity being performed. For example, most people naturally stand straight with a good posture when they consciously realize that their back is not straight. However, because of the countless daily distractions, this awareness fades and the appropriate posture for the given activity gradually deteriorates. If not corrected, the bad posture worsens over time and may lead to permanent deformation of the spine and back pain in the exemplary instance of improper posture for the activity of standing, sitting, or the deterioration of performance of the activity (such as a poor tennis service due to improper posture).

When a human mode is selected by the user, SCPMS 700 presents different activity options for a user to perform in conformity with best practices for best spine posture protection. A select activity window 702 presents a menu including a series of potential activities that can be performed by the user. Such activities include, but are not limited to stretching 704, sitting 706, sleeping 708 or performing an athletic activity 710.

SCPMS 700 allows users to orient (or position) their body to a proper posture for the given activity and save that proper posture as a preferred reference posture for that activity, and reminds users to always maintain the preferred posture if the body posture (position) deviates from it. For example, with the present disclosure, users may stand naturally straight and save that posture as preferred reference posture for standing, or stretching, and reminds the user to always maintain the preferred reference posture if the body posture deviates from it.

In one example, a user can select stretching activity 704 from select activity menu 702. In FIG. 7B, a series of postures 712 are presented that the user can either repeat, and save as proper default postures to be measured against later, or can have the option to download the series of postures, save as default and attempt to conform to them as part of performing the activity as further described in this disclosure.

One advantage of embodiments described in the present disclosure is that they allow a user to train the muscles to subconsciously hold that preferred reference posture, even after the present disclosure is no longer worn. With severe cases of deformity or a user new to a given activity, such as stretching, or another physical activity, such as tennis, and with the supervision of a physician, a physical therapist, or a trainer, users may consciously work the specific muscles using the present disclosure to incrementally and gradually correct their posture for the given activity. That is, in severe cases (or if the user is new to the activity) where the correction may be too drastic to achieve in one step, the present disclosure be used to gradually correct and train for good posture for a given activity in multiple steps. At every step, the appropriate posture is saved and the muscles are gradually trained to finally achieve correct posture for the given activity as will be further illustrated by FIGS. 9A-9H.

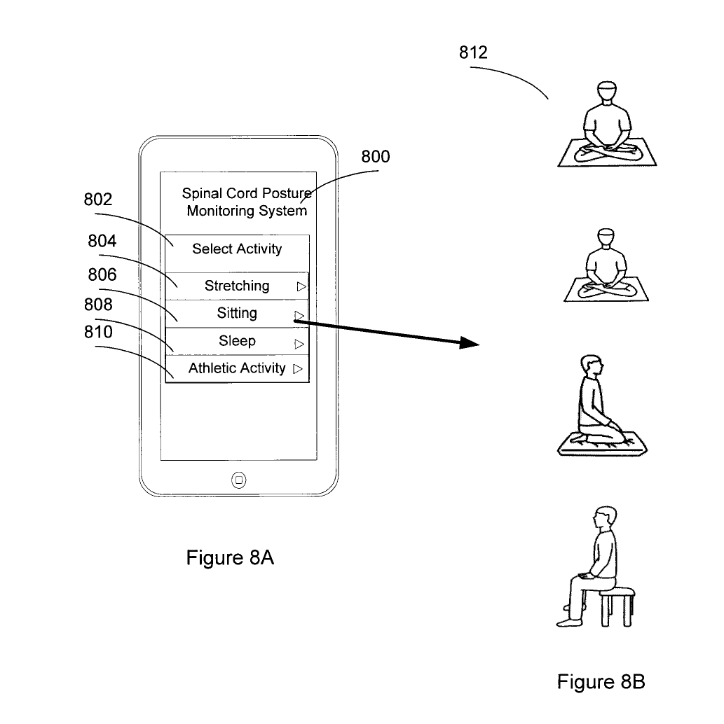

FIG. 8A is a sample graphical user interface displaying sitting activity options for a human associated with the spinal cord posture monitoring system according to an exemplary embodiment. FIG. 8B is a sample display of sitting activity options for a human associated with the spinal cord posture monitoring system. SCPMS 800 includes a select activity window 702 that presents a menu including a series of potential activities that can be performed by the user. Such activities include, but are not limited to stretching 804, sitting 806, sleeping 808 or performing an athletic activity 810. In one example, and as further illustrated by FIG. 7B, a user can select the sitting activity 808 and a series of pre-programmed sitting postures 812 are displayed. The displayed sitting postures 812 can be used beneficially in several applications. Firstly, the displayed sitting postures 812 can be used to guide the user to understand and learn the proper postures for the selected activity. For example, sitting on knees, chair or on a floor mat, may require the user to sit in certain manners and keep a specific spinal cord posture. Secondly, the displayed sitting postures 812 can also be used to gauge the conformity of the user to the specific activity posture. For example, if sitting on a chair sitting activity is selected, then the person's posture conformity is evaluated in comparison to the selected sitting activity. In this application, a selected sitting activity includes a pre-configured setting of back/spinal cord outline and the measured anterior/posterior positioning of the spinal cord posture measuring device (such as that illustrated in FIG. 5) will be measured against the pre-configured setting.

FIG. 9A is an illustration of a functional menu for measuring status and compliance of an individual wearing the spinal cord posture monitoring system while performing an athletic activity, according to an exemplary embodiment. In FIG. 9A, SCPMS 900 includes select activity menu 902, stretching menu 904, sitting menu 906, sleep menu 908 and athletic activity menu 910. Athletic activity menu includes a host of potential athletic activities that can be performed by the user, including, for example, tennis, football, swimming, volleyball, racquetball, and weight lifting. When a user selects a sport, for example tennis, the selection passes through a data analysis phase 912. Data analysis phase 912 configures the menu selections for the selected athletic activity and presents a secondary set of options for the user. Such options include the option to download default settings 914, input manual settings 916, track conformity index 918, review number of violations 920, suggested corrective measures 924 and suggested exercises 926. The features of each menu option and how they assist the user are described in greater detail in FIGS. 9B-9E below.

FIG. 9B is an illustration of a functional menu for downloading default settings to measure status and compliance of an individual wearing the spinal cord posture monitoring system according to an exemplary embodiment. In FIG. 9B, SCPMS 900 includes select activity menu 902, stretching menu 904, sitting menu 906, sleep menu 908 and athletic activity menu 910. Athletic activity menu includes a host of potential athletic activities that can be performed by the user, including, for example, tennis, football, swimming, volleyball, racquetball, and weight lifting. When a user selects a sport, for example tennis, the selection passes through a data analysis phase 912. Data analysis phase 912 configures the menu selections for the selected athletic activity and presents a secondary set of options for the user. Such options include the option to download default settings 914, input manual settings 916, track conformity index 918, review number of violations 920, suggested corrective measures 924 and suggested exercises 926.

When a user selects the option to download default settings 914, options to download default settings are presented to the user. Such options include, but are not limited to, downloading default settings from a remote server 930, downloading from a website 932, or downloading updates 934. Other options include downloading default settings from another user, downloading default settings from the user's doctor's office or recommended doctor site, and other options as would be recognized by one skilled in the art. Downloading default settings allows the user to download predetermined position templates that the user can adhere to, practice with and be compared and/or evaluated against. In one example, if a user knows that a specific website or server or remote location has new and improved default settings, the user may download the default settings for the first time or as updates to previously downloaded default settings. If a new yoga trend or new style of performing specific athletic activities is gaining popularity, a user may download default settings from a remote location. A user may also search for the remote location if preconfigured settings do not include a specific site the user would like to download from.

FIG. 9C is an illustration of a functional menu for inputting default settings to measure status and compliance of an individual wearing the spinal cord posture monitoring system according to an exemplary embodiment. SCPMS 900 further includes the capability for a user to input default settings manually 916 when the user either wishes to create his/her own template to follow, or cannot find an appropriate template to follow. In FIG. 9C, if a user selects to input their own default settings manually 916, SCPMS 900 presents several menu options to be configured, including input athletic activity style 940, record option/start recording 941, perform activity 942, stop recording 943, save manual recording as default setting 944 and compare manual recording against downloaded default settings 945.

Inputting athletic activity style 940 allows the user to input a style or stroke within an athletic activity. For example, if athletic activity 910 was selected to be tennis, an athletic activity stroke would be a tennis serve, a forehand, a back hand, volley or other stroke within tennis. Continuing on the tennis serve example, after selecting a tennis serve as the activity style, the user can record the activity. In one example, the user can set a predetermined time for starting and stopping the recording. In another example, the user can manually select to start the recording, 941, perform the activity 942, and stop the recording 943. The manual recording for the tennis serve activity can be saved as a default manual setting 944. The user has additional option to save the activity as a default manual setting so as to allow for additional future comparisons. Memory 306 can save many different types of default settings. For example, memory 306 can save downloaded default settings, or can save manual settings as default manual settings.

In another example, memory 306 can save both settings as downloaded default settings and manual default settings. Such saved data can be useful if the user wishes to compare, 945, manual recordings saved as default manual settings to downloaded default settings. The comparison allows the user to determine if their manually saved settings are within acceptable range to be used as default settings. This is useful if, for example, downloaded default settings do not take certain parameters, as person's height, weight, gender, or other parameters into account when determining setting default settings.

FIG. 9D is an illustration of a functional menu for tracking conformity index to measure status and compliance of an individual wearing the spinal cord posture monitoring system according to an exemplary embodiment. A user can track conformity of his activity with default settings. In doing so, the user can track conformity as an overall percentage, for example, the user may get a reading of 67% overall conformity, or 95% overall conformity. For more enhanced overview, a user can further select conformity breakdown option 952, to review a report of conformity for each sensor. For example, and referencing back to FIG. 2 and FIG. 5, a conformity breakdown can include a breakdown of each spherical encoder and the conformity percentage for each one. For example, a user may have several sensors placed within SCPMS assembly 200 such as 6 spherical encoders, or as further illustrated in FIG. 5, can be P₁-P₆. By viewing the conformity index breakdown, the user can determine if the entire spinal cord is non-conforming, or a specific segment is non-conforming.

FIG. 9E is an illustration of a functional menu for reviewing the number of violations to measure status and compliance of an individual wearing the spinal cord posture monitoring system according to an exemplary embodiment. After selecting to review the number of violations 920, the user can view a time stamp of every incident of non-conformity 962, a location stamp of every incident of non-conformity 964, and a detailed report of non-conformity incidents 966. For example, a user can view every time a non-conformity incident occurred. That is, if the user was sitting on a chair, or performing a stretching activity or an athletic activity, every time a non-conforming incident is triggered, the non-conforming incident is recorded. This included non-conforming incidents for any sensor within the system, such as sensors P₁-P₆.

The location stamp 964 includes the location of every non-conforming incident. For example, the user can view the exact geographic location where every non-conforming incident occurred. If the non-conforming incident occurred in an office, mall, basketball court, home, or any other location, the location is stamped and correlated with the time stamp associated with the incident. SCPMS 900 can use any one of or combinations of sensors associated with data processing and transmission processor 302 to measure and detect the location stamp. For example, SCPMS 900 can use position/GPS sensor 322 to determine the location of the incident. Alternatively, and for locations that are indoors, SCPMS 900 can use acceleration and or angular momentum sensors to determine the location of the incident.

The user can further view a detailed report 966 of non-conformity incidents. The user can generate the report by selecting the detail report option. In doing so the user can modify the generated report by viewing time and location stamps of every incident, nature of the incident, activity performed, and sensors involved in the non-conformity.

FIG. 9F is an illustration of a functional menu for reviewing the number of transmitted doctor pings to measure status and compliance of an individual wearing the spinal cord posture monitoring system according to an exemplary embodiment. SCPMS 900 can automatically transmit non-conformity reports to the user's registered medical professional, or alternatively can allow the user to dictate when the non-conformity reports are transmitted to the medical professional. In selecting to review number of doctor pings/notifications 922, the user can have the option to review the non-conformity report 970 that has either been sent to the medical professional or the non-conformity report that is ready to be sent. In one example, if the non-conformity report has not been sent, the user has the option to send the report himself by selecting that option. Furthermore, the user can also include additional information in the report, such as “experienced additional pain” or “repeating this activity causes continuous non-conformity” or the like.

The user can also list the number of reported incidents sent to the medical professional 972 for an selected period of time. For example, the user can view the number of non-conformity reports sent to the medical professional for a one hour period, 2 hour period, one day, one week and so on. Us user can also have the option to open a dialogue with someone from the medical professional's office. For example, the user can open a chat window and solicit advice from the medical office. Alternatively the user can transmit a text message or email to the doctor. In yet another example, the user can receive a message from the medical office indicating dangerous levels of non-conformity and request the user to schedule a visitation. These examples illustrate the wide array of possible uses and are not meant to be limiting as one of ordinary skill in the art may recognize.

FIG. 9G is an illustration of a functional menu for outputting suggested corrective measures for an individual wearing the spinal cord posture monitoring system according to an exemplary embodiment. A user the wishes to improve his/her posture, may do so by selecting the suggested corrective measures option 924. Suggested corrective measures can be stored on memory 306 or downloaded from a remote server location. Suggested corrective measures can include setting up a call/chat session with a medical professional 980, provide a list of potential medication recommendations 982, or provide a list of potential changes in activity performance and postures 984.

In one example, SCPMS 900 generates a chat session or call window for the user to engage a medical professional or a staff member of the medical office in order to make an appointment or discuss any non-conformity issues. Furthermore, a list of potential medication recommendations can be provided. The medication list may include over the counter medications or prescription medications. Prescription medications will need to be prescribed by the doctor, but the option provided can further advance the user's understanding of the issue and best medications to use. For example, if a prescription medication is recommended, the user can solicit the doctor's approval in providing the prescription medication, given that the doctor can view the non-conformity charts provided by SCPMS 900. Additionally, SCPMS 900 can provide a list of potential changes in activity 984. This option provides the user with a guidance as to how perform an activity differently. For example, and utilizing the earlier tennis serve example, assuming that SCPMS 900 registers a number of non-conformities for the user in performing a tennis serve. SCPMS 900 registers a location and time stamp for every activity, an overall percent conformity, and also detects which sensors are non-conforming to the default settings. Once registered, SCPMS 900 can illustrate how the athletic activity should be ideally performed. For example, if a tennis serve registers multiple non-conformities, SCPMS 900 can output default illustrations of how a proper tennis serve is performed. It can be presented in an array of different motion speeds to help the user fully view and understand the motion. SCPMS 900 can further list changes to more specific postures. For example, if a specific sensor or segment of sensors is non-conforming, then SCPMS 900 lists a series of suggested corrective postures to improve conformity.

FIG. 9H is an illustration of a functional menu for suggesting exercises for an individual wearing the spinal cord posture monitoring system according to an exemplary embodiment. SCPMS 900 may further suggest exercises 926 to the user. The suggested exercises can include, but are not limited to stretching exercises 990, measures to straighten back for a pre-determined period of time 991, bending exercises 992 and knee/torso exercises 993. When a user selects an exercises, SCPMS 900 records the activity as it is being performed and further can output a conformity index before and after the suggested exercises. This helps the user track the effectiveness of the exercises. The user may further download additional suggested exercises or manually input ones. SCPMS may further receive suggested exercises from a remote location, such as a medical office if the medical office determines a set of exercises is most suitable for the user.

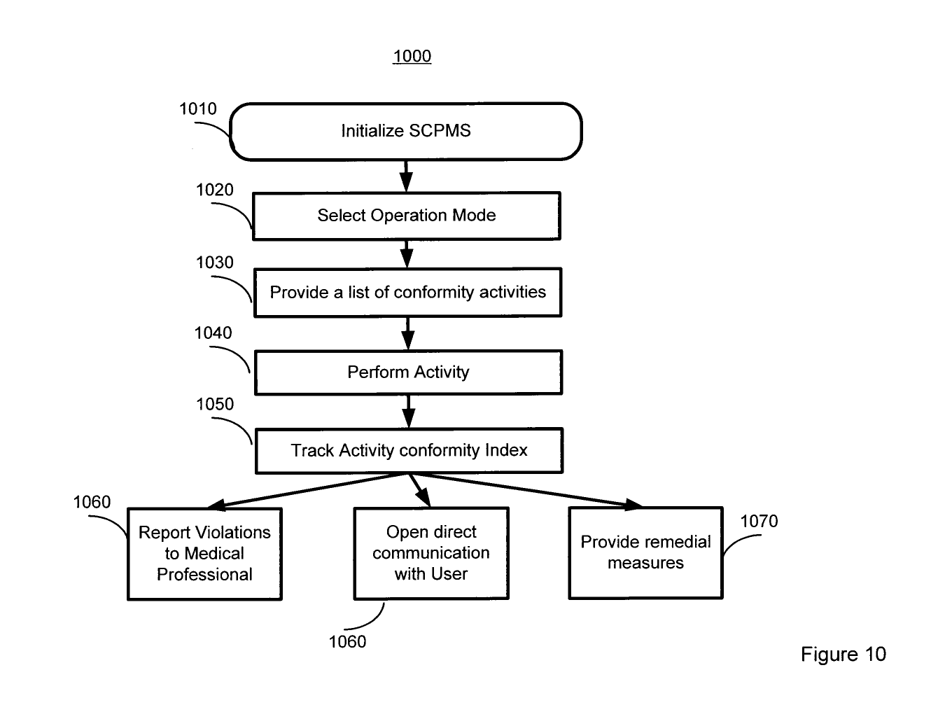

FIG. 10 is an illustration of an algorithmic flow diagram for measuring status and compliance method 1000 of an individual wearing the spinal cord posture monitoring system according to an exemplary embodiment. Method 1000 includes initializing SCPMS 1010, selecting an operational mode 1020, providing a list of conformity activities 1030, performing the activity 1040 tracking the activity 1050, reporting violations to medical professional 1060, opening direct communication between the user and medical professional 1070 and providing remedial measures 1080. Initializing SCPMS 1010 includes turning on the device and/or activating the monitoring user interface. The user can then select an operational mode 1020. Operational modes include selecting whether the user wishes to apply the SCPMS to a pet (Animal Mode) or a person (Human Mode). After selecting a mode, a conformity activity is provided 1030. Conformity activities include but are not limited to, stretching activities, sitting activities, sleeping activities and athletic activities. Athletic activities can further include sports activities, such as tennis, football, swimming, volleyball and weight lifting. Upon selecting the conformity activity, the activity is then performed by the user and a list of options is provided. Among the options, SCPMS can provide download default settings options, manually inputting settings options, tracking conformity index option, reviewing number of conformity violations options, reviewing number of medical professional pings options, suggested corrective measures and suggested exercise options. While performing the activity, the user can record and manipulate the recorded activity. As previously described, an activity is recorded by recording the measurements of each and every sensor in SCPMS system. Determining the conformity of each and every sensor allows SCPMS to track activity conformity 1050 against default settings. From that point, SCPMS can report violations to medical professional 1060. The violations reported can also be reported to the user. For example, if the user is sitting down and is hunched over, bending down, or slouching, SCPMS can provide a notification to the user of the non-conformity, including exact locations and sensors that are not conforming and suggests remedial measures 1070. In sever non-conformity incidences, or if the user is experiencing severe pain, SCPMS can open a direct communication between the user and a medical professional to further discuss alleviating the pain.

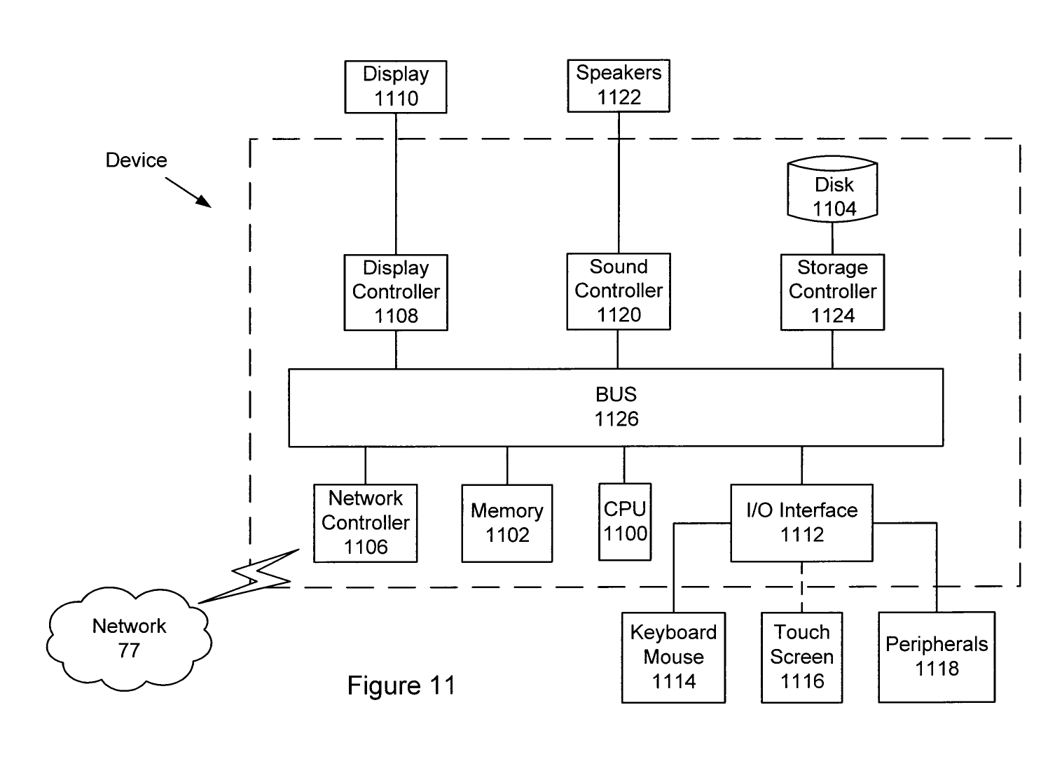

Next, a hardware description of a device according to exemplary embodiments illustrated in FIGS. 1-10 is described with reference to FIG. 11. In FIG. 11, the device includes a CPU 1100 which performs the processes described above. The process data and instructions may be stored in memory 1102. These processes and instructions may also be stored on a storage medium disk 1104 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device communicates, such as a server or computer.

Further, the present advancements may be provided as a utility application, background daemon, or component of an operating system, or combination thereof, executing in conjunction with CPU 1100 and an operating system such as Microsoft Windows 7, UNIX, Solaris, LINUX, Apple MAC-OS and other systems known to those skilled in the art.

CPU 1100 may be a Xenon or Core processor from Intel of America or an Opteron processor from AMD of America, or may be other processor types that would be recognized by one of ordinary skill in the art. Alternatively, the CPU 1100 may be implemented on an FPGA, ASIC, PLD or using discrete logic circuits, as one of ordinary skill in the art would recognize. Further, CPU 1100 may be implemented as multiple processors cooperatively working in parallel to perform the instructions of the inventive processes described above.

The device in FIG. 11 also includes a network controller 1106, such as an Intel Ethernet PRO network interface card from Intel Corporation of America, for interfacing with network 77. As can be appreciated, the network 77 can be a public network, such as the Internet, or a private network such as an LAN or WAN network, or any combination thereof and can also include PSTN or ISDN sub-networks. The network 77 can also be wired, such as an Ethernet network, or can be wireless such as a cellular network including EDGE, 3G and 4G wireless cellular systems. The wireless network can also be Wi-Fi, Bluetooth, or any other wireless form of communication that is known.

The device further includes a display controller 1108, such as a NVIDIA GeForce GTX or Quadro graphics adaptor from NVIDIA Corporation of America for interfacing with display 1110, such as a Hewlett Packard HPL2445w LCD monitor. A general purpose I/O interface 1112 interfaces with a keyboard and/or mouse 1114 as well as a touch screen panel 1116 on or separate from display 1110. General purpose I/O interface also connects to a variety of peripherals 1118 including printers and scanners, such as an OfficeJet or DeskJet from Hewlett Packard.

A sound controller 1120 is also provided in the device, such as Sound Blaster X-Fi Titanium from Creative, to interface with speakers/microphone 1122 thereby providing sounds and/or music.

The general purpose storage controller 1124 connects the storage medium disk 1104 with communication bus 1126, which may be an ISA, EISA, VESA, PCI, or similar, for interconnecting all of the components of the device. A description of the general features and functionality of the display 1110, keyboard and/or mouse 1114, as well as the display controller 1108, storage controller 1124, network controller 1106, sound controller 1120, and general purpose I/O interface 1112 is omitted herein for brevity as these features are known.

Thus, the foregoing discussion discloses and describes exemplary embodiments of the present disclosure for clarity. As will be understood by those skilled in the art, the present invention may be embodied in other specific forms without departing from the spirit or essential characteristics thereof and aspects of the exemplary embodiments described herein may be combined differently to form additional embodiments or omitted. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

Embodiments of the present disclosure provide a posture training device that corrects posture in relation to a reference. More specifically, the present disclosure provides a posture training and monitoring device that corrects posture in relation to other body parts, such as a neck as well as a true earth vertical. Accordingly, the present invention provides sensory indication modules that are intimately associated with a surface for detection of angles relative to true vertical and acceleration, and includes feedback indicators for communicating information in relation to the detected angles of the surface with which the sensory indication modules are associated, with the user. The present invention combines the high performance sensors with electronics to create a posture correction training and monitoring device that is easy to wear on a daily basis and provides true posture correction for users for a given activity. The present disclosure further provides at least one control module for communicating command and control instructions with the sensory indication modules, including the activation of the feedback indicators (or stimulations) for correction of posture for a given activity. The present disclosure makes use of multiple feedback indicators (or stimulation mechanisms) that are placed at each measurement location to accurately alert users of the specific location of the body that needs correction for the given activity, and instructs the users with respect to the manner of correcting the posture. For example, a user may be in non-conformity with regards to a specific activity being performed. A user interface may visually illustrate the non-conformity in accordance with an overlay of the spinal cord posture diagram. In addition or in the alternative, the sensor devices can be further outfitted with feedback indications, such as stimulations at the non-conforming posture measured by a spherical encoder.

1. A spinal cord posture monitoring system comprising:
a wearable measurement device configured to house a plurality of mechanical assembly segments, wherein each one of the plurality of mechanical assembly segments includes
a spherical encoder configured to detect a position parameter and a motion parameter about three mutual orthogonal axes,
a hollow tube configured to be affixed on one end to another one of the plurality of mechanical assembly segments, and
a connector link operatively connected to the spherical encoder on one end and connected to the hollow tube on the opposite end of the hollow tube that is opposite the one end; and
a communication circuit configured to communicate position and motion parameters as posture data for each spherical encoder of the wearable measurement device to a mobile application on a mobile device.

2. The spinal cord posture monitoring system of claim 1, wherein at least one spherical encoder is configured to map a location of at least one vertebrae of a spinal cord.

3. The spinal cord posture monitoring system of claim 1, further comprising:
at least one stretching sensor configured to capture stretching data of the wearable measurement device in an anterior and in a posterior plane.

4. The spinal cord posture monitoring system of claim 1, further comprising:
a neck sensor configured to operatively connect to the wearable measurement device and to measure neck motion and neck orientation with regard to the spinal cord.

5. The spinal cord posture monitoring system of claim 1, wherein the communication circuit is configured to:
record incidents of a non-conformity for each mechanical assembly segment; and
record a time stamp and a geographic location stamp associated with the incidents of non-conformity.

6. The spinal cord posture monitoring system of claim 1, wherein the mobile device is configured to operate in an animal monitoring mode and a human monitoring mode.

7. The spinal cord posture monitoring system of claim 1, wherein an activity for which conformity is measured and recorded is selectable on the mobile application.

8. The spinal cord posture monitoring system of claim 7, wherein the activity includes stretching, sitting, sleeping and performing an athletic activity.

9. The spinal cord posture monitoring system of claim 8, wherein the athletic activity further includes tennis, football, swimming volleyball and weight lifting.

10. The spinal cord posture monitoring system of claim 9, wherein after selecting an activity, the mobile application outputs a capability to download default settings from a remote location or record default manual settings.

11. The spinal cord posture monitoring system of claim 10, wherein default manual settings include at least one set of posture data, associated with the select activity, for the at least one of the mechanical assembly segments.

12. The spinal cord posture monitoring system of claim 11, wherein the spinal cord posture monitoring system compares recorded default manual settings to downloaded default settings to measure accuracy of recorded default manual settings.

13. The spinal cord posture monitoring system of claim 10, wherein the mobile application is configured to provide a review of the number of non-conforming violations recorded by the comfort level monitoring system within a predetermined period of time.

14. The spinal cord posture monitoring system of claim 10, wherein the mobile application is further configured to automatically transmit a notification to a medical facility indicating an occurrence of a non-conforming posture incident.

15. The spinal cord posture monitoring system of claim 14, wherein the mobile application is further configured to suggest corrective measures to correct the non-conforming posture incident.

16. The spinal cord posture monitoring system of claim 16, wherein the corrective measures include a list of medication recommendations, a list of changes in posture, or initiating direct communications with the medical facility.

17. The spinal cord posture monitoring system of claim 1, further comprising a vibrating sensor configured to vibrate at a location where a posture non-conformity is detected.

18. The spinal cord posture monitoring system of claim 1, wherein the mobile application is configured to display non-conforming activities to provide an alert of the non-conforming activities.

19. A spinal cord posture monitoring method comprising:
placing a sensor for each vertebrae of a spinal cord to mimic an alignment of the spinal cord;
selecting an activity to be performed by a person;
determining default conformity settings for the selected activity;
generating a conformity index for the selected activity based on a comparison between measured alignment data at each sensor and the default conformity settings;
generating a time stamp and location stamp report indicative of measured non-conformity incidents; and
generating an alert to remedy the non-conformity.

20. A non-transitory computer readable medium having computer-readable instructions thereon which when executed by a computer cause the computer to perform a method according to claim 19.
